(19) United States
(12) Patent Application Publication
(10) Pub. No.: US 2025/0170140 A1
(43) Pub. Date: May 29, 2025

(54) PHARMACEUTICAL COMPOSITION FOR PREVENTING OR TREATING CANCER COMPRISING CARNITINE ACYLCARNITINE CARRIER INHIBITOR AND PEROXISOMAL BETA OXIDATION INHIBITOR

(71) Applicant: NEW CANCER CURE-BIO CO., LTD., Goyang-si, Gyeonggi-do (KR)

(72) Inventor: Soo Youl KIM, Goyang-si (KR)

(73) Assignee: NEW CANCER CURE-BIO CO., LTD., Goyang-si, Gyeonggi-do (KR)

(21) Appl. No.: 18/725,204

(22) PCT Filed: Dec. 22, 2022
(86) PCT No.: PCT/KR2022/021020

§ 371 Date: Jun. 28, 2024

(30) Foreign Application Priority Data
Dec. 28, 2021 (KR) 10-2021-0189971
Dec. 21, 2022 (KR) 10-2022-0180501

(51) Int. Cl. A61K 31/5415; A61K 31/4439; A61K 45/06; A61P 35/00

(52) U.S. Cl. CPC A61K 31/5415; A61K 31/4439; A61K 45/06; A61P 35/00

(57) ABSTRACT

The present invention relates. to a pharmaceutical composition for preventing or treating cancer, comprising a carnitine acylcarnitine carrier (CAC) inhibitor and a peroxisomal beta oxidation inhibitor. In the present invention, co-administration of a carnitine acylcarnitine carrier inhibitor and a peroxisome beta-oxidation inhibitor not only significantly reduced cancer cell growth compared to each drug alone, but also confirmed the synergistic anti-cancer effect of co-administration. Therefore, the composition comprising a carnitine acylcarnitine carrier inhibitor and a peroxisome beta-oxidation inhibitor of the present invention can be provided as an effective combination anti-cancer agent.

### TECHNICAL FIELD

The present invention relates to a pharmaceutical composition for preventing or treating cancer, comprising a carnitine acylcarnitine carrier (CAC) inhibitor and a peroxisomal beta oxidation inhibitor.

### BACKGROUND ART

While normal cells are capable of regular, resilient proliferation and suppression as needed, cancer cells have unrestricted proliferation, resulting in a cell mass composed of undifferentiated cells, also known as a tumor. These cancer cells infiltrate the surrounding tissues and metastasize to other organs of the body, causing severe suffering and eventually death. Despite medical advances, the number of cancer patients in Korea has continued to increase, increasing by about 44% in the last decade, and the market for anti-cancer drugs has also increased internationally, with a reported annual value of about $100 billion.

The first generation of anti-cancer drugs are chemical anti-cancer drugs, the second generation are targeted anti-cancer drugs, and immune anti-cancer drugs are being developed as the third generation of anti-cancer drugs to overcome their side effects. However, the biggest problem in current cancer treatment is cancer recurrence because it is difficult to target specific cancers due to various mutations in cancer, and it is not uncommon for recurrent cancers to develop resistance to the anticancer drugs used in the treatment process. As a result, even after the primary cancer is treated, patients often die from metastatic and recurrent cancers. Therefore, in order to improve the effectiveness of anticancer drugs, strategies have been proposed to combine anticancer drugs in combination.

Meanwhile, omeprazole, a carnitine acylcarnitine carrier (CAC) inhibitor, is a proton-pump inhibitor that has been shown to be effective in the treatment of gastroesophageal reflux disease, peptic ulcers, erosive esophagitis, or eosinophilic esophagitis. A recent study showed that proton-pump inhibitors, including omeprazole, inhibit the activity of Fatty Acid Synthase (FASN), which helps cancer cells produce fatty acids that are key to their survival, and induce cancer cell death with minimal effect on non-cancerous cells (Walsh et al., Journal of Experimental & Clinical Cancer Research, 34:93, 2015).

Thioridazine, a peroxisomal beta oxidation inhibitor, is a 10-[2-(1-methyl-2-piperidyl)ethyl]-2-(methylthio)phenothiazine compound that was developed as a first-generation antipsychotic and has been used to treat psychotic disorders such as psychosis and schizophrenia, etc. A recent novel use of thioridazine has been its antimicrobial activity against drug-resistant microorganisms such as Mycobacterium tuberculosis.

However, the combined administration of a carnitine acylcarnitine carrier inhibitor (omeprazole) and a peroxisome beta-oxidation inhibitor (thioridazine) has not been disclosed.

### DISCLOSURE

### Technical Problem

Accordingly, the present inventors have made a conscientious effort to provide a combination anticancer drug that can significantly inhibit cancer cells. We have confirmed that the combination treatment of a carnitine acylcarnitine carrier inhibitor and a peroxisome beta-oxidation inhibitor significantly increases the inhibitory effect on cancer cells compared to the treatment of each drug alone, and have completed the present invention.

Accordingly, it is an object of the present invention to provide a pharmaceutical composition for the prevention or treatment of cancer comprising a carnitine acylcarnitine carrier inhibitor and a peroxisome beta-oxidation inhibitor.

### Technical Solution

To fulfill the purposes described above, the present invention provides a pharmaceutical composition for the prevention or treatment of cancer comprising a carnitine acylcarnitine carrier (CAC) inhibitor and a peroxisomal beta oxidation inhibitor.

The present invention also provides an anti-cancer adjuvant comprising carnitine acylcarnitine carrier inhibitors and peroxisome beta-oxidation inhibitors.

The present invention also provides a method of preventing or treating cancer, comprising the step of administering or taking to a subject a composition comprising a carnitine acylcarnitine carrier (CAC) inhibitor and a peroxisomal beta oxidation inhibitor.

The present invention also provides a use of a composition comprising a carnitine acylcarnitine carrier (CAC) inhibitor and a peroxisomal beta oxidation inhibitor for the prevention or treatment of cancer.

According to a preferred embodiment of the present invention, the carnitine acylcarnitine carrier inhibitor may be one or more selected from the group consisting of omeprazole (KN510), lansoprazole (KN511), pantoprazole (KN512) and pharmaceutically acceptable salts thereof, wherein the omeprazole may be the compound represented by Formula 1 below:

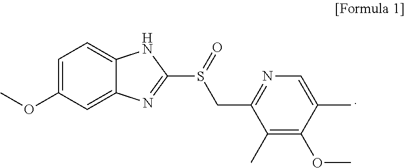

According to another preferred embodiment of the present invention, the peroxisome beta-oxidation inhibitor may be thioridazine (KN714) or a pharmaceutically acceptable salt thereof, wherein the thioridazine may be a compound represented by Formula 2 below:

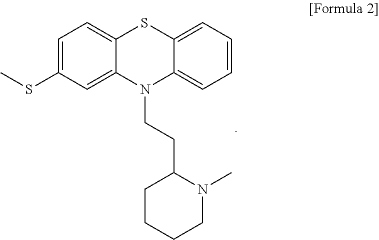

According to another preferred embodiment of the present invention, the carnitine acylcarnitine carrier inhibitor and the peroxisome beta-oxidation inhibitor may be included in a concentration ratio of 100:1 to 1:100, preferably in a concentration ratio of 100:1 to 10:1.

According to another preferred embodiment of the present invention, the carnitine acylcarnitine carrier inhibitor and the peroxisome beta-oxidation inhibitor may be administered sequentially or simultaneously.

According to another preferred embodiment of the present invention, the cancer may be any one or more cancers selected from the group consisting of breast cancer, colon cancer, glioblastoma, liver cancer, leukemia, melanoma, lung cancer, ovarian cancer, prostate cancer, pancreatic cancer, renal cell carcinoma, and gastric cancer.

According to another preferred embodiment of the present invention, the pharmaceutical composition or anti-cancer adjuvant may further comprise an additional anti-cancer agent, wherein the anti-cancer agent may be one or more anti-cancer agents selected from one or more metabolism inhibitors selected from the group consisting of Irinotecan, Fluorouracil (5-FU), Paclitaxel, Gemcitabine, Cisplatin, Vermurafenib, and pharmaceutically acceptable salts thereof; and one or more tumor immunosuppressive agents selected from the group consisting of Pembrolizumab, Nivolumab, Atezolizumab, Ipilimumab, and Durvalumab.

### Advantageous Effects

In the present invention, co-administration of a carnitine acylcarnitine carrier inhibitor and a peroxisome beta-oxidation inhibitor not only significantly reduced cancer cell growth compared to each drug alone, but also confirmed the synergistic anti-cancer effect of co-administration. Therefore, the composition comprising a carnitine acylcarnitine carrier inhibitor and a peroxisome beta-oxidation inhibitor of the present invention can be provided as an effective combination anti-cancer agent.

### DESCRIPTION OF DRAWINGS

FIG. 1 shows data on the degree of cancer cell growth when pancreatic cancer cell lines were treated with omeprazole (KN510), thioridazine (KN714), or trimetazidine (KN713) at different concentrations.

FIG. 2 shows data on the degree of cancer cell growth when treated with (A) omeprazole (KN510) and thioridazine (KN714) and (B) omeprazole (KN510) and trimetazidine (KN713).

FIG. 3 shows data on the degree of cancer cell growth when breast cancer cell lines were treated with omeprazole (KN510) and thioridazine (KN714).

FIG. 4 shows data on the degree of cancer cell growth when colon cancer cell lines were treated with omeprazole (KN510) and thioridazine (KN714).

FIG. 5 shows data on the degree of cancer cell growth when glioblastoma cell lines were treated with omeprazole (KN510) and thioridazine (KN714).

FIG. 6 shows data on the degree of cancer cell growth when liver cancer cell lines were treated with omeprazole (KN510) and thioridazine (KN714).

FIG. 7 shows data on the degree of cancer cell growth when leukemia cell lines were treated with omeprazole (KN510) and thioridazine (KN714).

FIG. 8 shows data on the degree of cancer cell growth when melanoma cell lines were treated with omeprazole (KN510) and thioridazine (KN714).

FIG. 9 shows data on the degree of cancer cell growth when lung cancer cell lines were treated with omeprazole (KN510) and thioridazine (KN714).

FIG. 10 shows data on the degree of cancer cell growth when ovarian cancer cell lines were treated with omeprazole (KN510) and thioridazine (KN714).

FIG. 11 shows data on the degree of cancer cell growth when prostate cancer cell lines were treated with omeprazole (KN510) and thioridazine (KN714).

FIG. 12 shows data on the degree of cancer cell growth when pancreatic cancer cell lines were treated with omeprazole (KN510) and thioridazine (KN714).

FIG. 13 shows data on the degree of cancer cell growth when renal cell carcinoma cell lines were treated with omeprazole (KN510) and thioridazine (KN714).

FIG. 14 shows data on the degree of cancer cell growth when gastric cancer cell lines were treated with omeprazole (KN510) and thioridazine (KN714).

### MODE OF THE INVENTION

The present invention will be described in more detail below:

The present inventors combined several fatty acid oxidation metabolism inhibitors in order to provide an anti-cancer drug that can significantly inhibit cancer cells, and surprisingly confirmed that co-administration of a carnitine acylcarnitine carrier inhibitor and a peroxisome beta-oxidation inhibitor not only significantly reduced cancer cell growth compared to each drug alone, but also confirmed the synergistic anti-cancer effect of co-administration.

Accordingly, in one aspect, the present invention relates to a pharmaceutical composition for preventing or treating cancer comprising a carnitine acylcarnitine carrier (CAC) inhibitor and a peroxisomal beta oxidation inhibitor.

In another aspect, the present invention relates to an anticancer adjuvant comprising a carnitine acylcarnitine carrier inhibitor and a peroxisome beta-oxidation inhibitor.

In the present invention, the carnitine acylcarnitine carrier inhibitor may be one or more selected from the group consisting of omeprazole (KN510), lansoprazole (KN511), pantoprazole (KN512) and pharmaceutically acceptable salts thereof, wherein the omeprazole may be the compound represented by Formula 1 below:

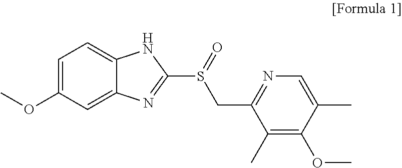

In the present invention, the peroxisome beta-oxidation inhibitor may be thioridazine (KN714) or a pharmaceutically acceptable salt thereof, wherein the thioridazine may be a compound represented by Formula 2 below:

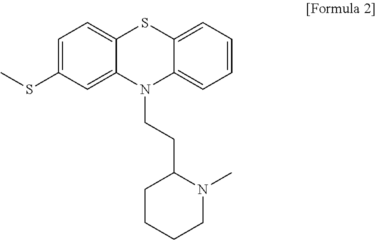

In the present invention, the carnitine acylcarnitine carrier inhibitor and the peroxisome beta-oxidation inhibitor may be included in a concentration ratio of 100:1 to 1:100, preferably in a concentration ratio of 100:1 to 10:1.

In the present invention, the carnitine acylcarnitine carrier inhibitor and the peroxisome beta-oxidation inhibitor may be administered sequentially or simultaneously.

In the present invention, the cancer may be any one or more cancers selected from the group consisting of breast cancer, colon cancer, glioblastoma, liver cancer, leukemia, melanoma, lung cancer, ovarian cancer, prostate cancer, pancreatic cancer, renal cell carcinoma, and gastric cancer.

Omeprazole (KN510) inhibits a carnitine acylcarnitine carrier in mitochondria, and thioridazine (KN714) inhibits beta-oxidation in peroxisomes, thereby inhibiting fatty acid oxidation, which is key to cancer cell survival. Therefore, in the present invention, co-administration of omeprazole (KN510) and thioridazine (KN714) can more effectively inhibit the fatty acid oxidation metabolism of cancer cells, resulting in synergistic anti-cancer effects.

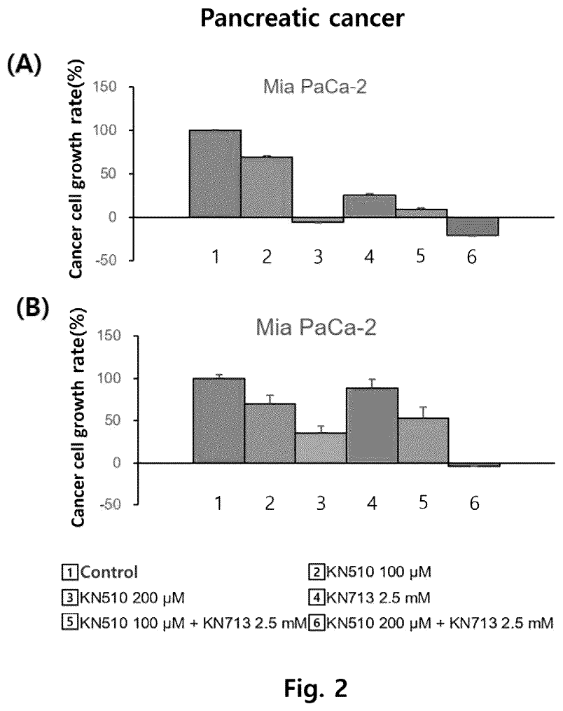

In a specific embodiment of the present invention, a pancreatic cancer cell line (MIA PaCa-2) was treated with omeprazole (KN510) and thioridazine (KN714) individually or in combination, and it was found that the combination of omeprazole (KN510) and thioridazine (KN714) significantly increased the cancer cell-killing effect compared to the single treatment group, as shown in FIG. 2.

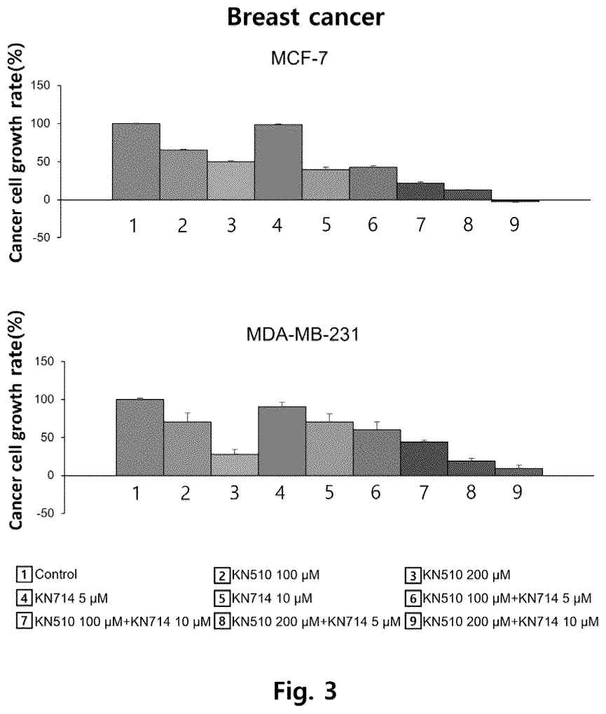

In another specific embodiment of the present invention, omeprazole (KN510) and thioridazine (KN714) were administered individually or in combination to breast cancer, colon cancer, glioblastoma, liver cancer, leukemia, melanoma, lung cancer, ovarian cancer, prostate cancer, pancreatic cancer, renal cell carcinoma, and stomach cancer cell lines, respectively. As a result, it was confirmed that the cancer cell killing effect was significantly increased when omeprazole (KN510) and thioridazine (KN714) were administered in combination compared to the single administration group (FIGS. 3 to 14). In addition, it was confirmed that combined administration of omeprazole (KN510) and chlorpromazine (KN306) showed synergistic anticancer effect.

Furthermore, in another specific embodiment of the present invention, the combination of omeprazole (KN510) and thioridazine (KN714) of the present invention was superior in killing cancer cells compared to the combination of omeprazole (KN510) and trimetazidine (KN713), a 3-ketoacyl CoA thiolase inhibitor in vitro.

In the present invention, the pharmaceutical composition or anti-cancer adjuvant may further comprise an additional anti-cancer agent, wherein the anti-cancer agent may be one or more anti-cancer agents selected from one or more metabolism inhibitors selected from the group consisting of Irinotecan, Fluorouracil (5-FU), Paclitaxel, Gemcitabine, Cisplatin, Vermurafenib, and pharmaceutically acceptable salts thereof; and one or more tumor immunosuppressive agents selected from the group consisting of Pembrolizumab, Nivolumab, Atezolizumab, Ipilimumab, and Durvalumab.

The pharmaceutical composition of the present invention can be in a variety of oral or parenteral formulations. The compositions may be formulated using one or more buffers (e.g., saline or PBS), antioxidants, bacteriostatic agents, chelating agents (e.g., EDTA or glutathione), fillers, bulking agents, binders, auxiliaries (e.g., aluminum hydroxide), suspending agents, thickening agents wetting agents, disintegrating agents or surfactants, diluents, or excipients.

Solid dosage forms for oral administration include tablets, pills, powders, granules, capsules, and the like, wherein one or more compounds are combined with at least one excipient, such as starch (including corn starch, wheat starch, rice starch, potato starch, and the like), calcium carbonate, sucrose, lactose, dextrose, sorbitol, mannitol, mannitol, xylitol, erythritol maltitol, cellulose, methyl cellulose, sodium carboxymethylcellulose and hydroxypropylmethyl-cellulose, or gelatin. For example, tablets or sugar-coated tablets can be obtained by mixing the active ingredient with solid excipients, grinding it, adding suitable excipients and processing it into a granular mixture.

In addition to simple excipients, lubricants such as magnesium stearate, talc, etc. are also used. Liquid preparations for oral administration include suspensions, solutions, emulsions, or syrups, which may contain a variety of excipients, such as wetting agents, sweeteners, flavors, or preservatives, in addition to the commonly used simple diluents of water and liquid paraffin. In addition, in some cases, cross-linked polyvinylpyrrolidone, agar, alginate or sodium alginate may be added as a disintegrating agent, and may further include anti-flocculants, lubricants, wetting agents, flavors, emulsifiers, and preservatives.

Formulations for parenteral administration include sterile aqueous solutions, non-aqueous solutions, suspensions, emulsions, lyophilizates, or suppositories. Non-aqueous solvents and suspensions may include propylene glycol, polyethylene glycol, vegetable oils such as olive oil, and injectable esters such as ethylolate. As a base for suppositories, witepsol, macrogol, tween 61, cacao paper, laurin paper, glycerol, gelatin, etc. can be used.

The pharmaceutical composition of the present invention may be administered orally or parenterally and, when administered parenterally, may be formulated according to methods known in the art in the form of an injectable for topical use; intraperitoneal, rectal, intravenous, intramuscular, subcutaneous, intrauterine, intrathecal, or intracerebral injection.

In the case of the injectable, it must be sterile and protected from contamination by microorganisms such as bacteria and fungi. Examples of suitable carriers for injectables include, but are not limited to, solvents or dispersion media comprising water, ethanol, polyols (e.g., glycerol, propylene glycol, and liquid polyethylene glycol), mixtures thereof, and/or vegetable oils. More preferably, suitable carriers include Hanks' solution, Ringer's solution, phosphate buffered saline (PBS) containing triethanolamine, or isotonic solutions such as sterile water for injection, 10% ethanol, 40% propylene glycol, and 5% dextrose. To protect the injectable from microbial contamination, the injectable may additionally contain various antibacterial and antifungal agents such as parabens, chlorobutanol, phenols, sorbic acid, thimerosal, and the like. In addition, the injectable may further comprise an isotonicizing agent such as sugar or sodium chloride in most cases.

The pharmaceutical composition of the present invention can be administered in a pharmaceutically effective amount. A pharmaceutically effective amount means an amount sufficient to treat a condition with a reasonable benefit/risk ratio applicable to medical treatment, and the effective dose level may be determined based on factors including the type and severity of the patient's condition, the activity of the drug, sensitivity to the drug, time of administration, route of administration and elimination rate, duration of treatment, concomitant medications, and other factors well known in the medical field. The composition of the present invention may be administered as individual therapeutic agents or in combination with other therapeutic agents, may be administered sequentially or simultaneously with conventional therapeutic agents, and may be administered in single or multiple doses, i.e., the total effective amount of the composition of the present invention may be administered to the patient in a single dose, or may be administered in a fractionated treatment protocol in which multiple doses are administered over a longer period of time. Taking all of the above factors into account, it is important to administer an amount that will provide the maximum benefit in the least amount of time without side effects, which can be readily determined by those skilled in the art.

The preferred dosage of the composition depends on the condition of the patient, body weight, severity of the disease, drug form, route of administration and duration, but may be suitably selected by those skilled in the art, for example from 0.0001 to 2,000 mg/kg per day, more preferably from 0.001 to 2,000 mg/kg per day. The dose may be administered once daily or may be divided into several doses. However, the above dosages do not limit the scope of the invention.

The pharmaceutical composition of the present invention can be used alone or in combination with surgery, radiation therapy, hormone therapy, chemotherapy, and methods using biological response modifiers.

The anticancer adjuvant of the present invention refers to any form of anti-cancer agent intended to increase the anti-cancer effect of an anti-cancer agent or to suppress or improve the side effects of an anti-cancer agent. The anti-cancer adjuvant of the present invention can be administered in combination with various types of anti-cancer drugs or anti-cancer adjuvants, and when administered in combination, the anti-cancer drug can be administered at a lower level than the conventional anti-cancer drug dosage, but still show an equivalent level of anti-cancer effect, thus enabling safer anti-cancer treatment.

The route of administration of the anticancer adjuvant may be by any conventional route as long as it can reach the target tissue. The anticancer adjuvant of the present invention may be administered by, but is not limited to, intraperitoneal, intravenous, intramuscular, subcutaneous, oral, intrapulmonary, or rectal administration, depending on the intended use. Furthermore, the anticancer adjuvant may be administered by any device capable of transporting the active substance to the target cell.

The anticancer adjuvant of the present invention can be preferably formulated as an anticancer adjuvant by including one or more pharmaceutically acceptable carriers in addition to the active ingredient for administration. Carriers, excipients, or diluents that may be included in the anticancer therapeutic adjuvants of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils, including but not limited to.

The anticancer adjuvant of the invention may be a formulation for oral or parenteral administration, and the description of the formulation is hereby superseded by the description of the pharmaceutical composition.

The present invention will now be described in more detail with reference to the following examples. These embodiments are for the purpose of illustrating the invention only, and it will be apparent to one of ordinary skill in the art that the scope of the invention is not to be construed as limited by these embodiments.

### Example 1: Confirmation of Anticancer Activity by Co-Administration of a Carnitine Acylcarnitine Carrier Inhibitor and a Peroxisome Beta-Oxidation Inhibitor

To determine the effect of the combination treatment of omeprazole (KN510), a carnitine acylcarnitine carrier inhibitor, and thioridazine (KN714), a peroxisome beta-oxidation inhibitor, on cancer cell growth, the extent of cancer cell death was determined using the SRB assay (Sulforhodamine B colorimetric assay), and the degree of cancer cell growth was analyzed relative to the control (100%). As a comparison, omeprazole (KN510) and trimetazidine (KN713), a 3-ketoacyl CoA thiolase inhibitor, were co-treated.

### 1-1: Measuring the GI₅₀ Value

First, a pancreatic cancer cell line, MIA PaCa-2, was prepared, and cells (100 μl) were inoculated into 96-well cell culture plates at plating densities ranging from 5,000 to 20,000 cells/well, depending on the doubling time of each cell line. After cell inoculation, 96-well cell culture plates were incubated for 24 hours before the addition of experimental drugs. MIA PaCa2 cells were treated with omeprazole (KN510) at concentrations of 10⁻⁸ M, 10⁻⁷ M, 10⁻⁶ M, 10⁻⁵ M, 10⁻⁴ M, and 10⁻³ M, thioridazine (KN714) at concentrations of 10⁻¹⁰ M, 10⁻⁹ M, 10⁻⁸M, 10⁻⁷ M, 10⁻⁶ M, and 10⁻⁵ M, and trimetazidine (KN713) at concentrations of 10⁻⁷ M, 10⁻⁶ M, 10⁻⁵ M, 10⁻⁴ M, 10⁻³ M, and 10⁻² M, respectively.

The cells were then incubated in a CO₂ incubator for 48 hours, after which the assay was terminated by the addition of cold TCA. Cells were fixed in situ by gently adding 50 μl of cold 50% (w/v) TCA (final concentration: 10% TCA) and incubated at 4° C. for 60 min. The supernatant was discarded, and the plate was washed five times with distilled water and air dried. Sulforhodamine B (SRB) solution (100 μl) of 0.4% (w/v) in 1% acetic acid was added to each well, and the plate was left at room temperature for 10 min. After staining, unbound dye was removed by washing five times with 1% acetic acid, and the plate was air-dried. The bound dye was then solubilized with 10 mM trizma base, and the absorbance was recorded at 515 nm using an automated plate reader.

TABLE 1
Cancer cell growth rate according to concentration of omeprazole (KN510),
thioridazine (KN714), and trimetazidine (KN713), respectively

| Omeprazole (KN510) | | | Thioridazine (KN714) | | | Trimetazidine (KN713) | | |
|---|---|---|---|---|---|---|---|---|
| Conc. | Average (%) | STD (±) | Conc. | Average (%) | STD (±) | Conc. | Average (%) | STD (±) |
| 0M | 100.00 | 1.61 | 0M | 100.00 | 1.74 | 0M | 100.00 | 0.61 |
| 10⁻⁸M | 101.17 | 2.38 | 10⁻¹⁰M | 97.11 | 1.97 | 10⁻⁷M | 97.85 | 2.33 |
| 10⁻⁷M | 97.84 | 0.59 | 10⁻⁹M | 97.03 | 2.16 | 10⁻⁶M | 97.75 | 2.56 |
| 10⁻⁶M | 93.66 | 1.01 | 10⁻⁸M | 90.56 | 0.53 | 10⁻⁵M | 94.52 | 1.62 |
| 10⁻⁵M | 94.56 | 1.99 | 10⁻⁷M | 95.13 | 1.96 | 10⁻⁴M | 95.50 | 2.32 |
| 10⁻⁴M | 68.78 | 0.50 | 10⁻⁶M | 94.73 | 1.20 | 10⁻³M | 95.03 | 1.43 |
| 10⁻³M | −17.07 | 0.37 | 10⁻⁵M | −10.55 | 2.09 | 10⁻²M | −29.68 | 2.48 |

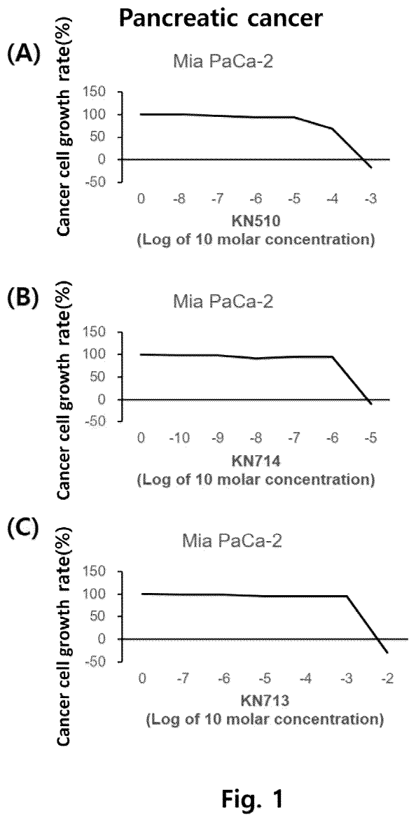

To determine the appropriate dosage concentrations for omeprazole (KN510) and thioridazine (KN714), the GI₅₀ (Half maximal growth inhibition concentration) values were determined based on the results in Table 1 and FIG. 1 below, and the GI₅₀ value for omeprazole (KN510) was 117.3 μM, the GI₅₀ value for thioridazine (KN714) was 1.9 μM, and the GI₅₀ value for trimetazidine (KN713) was 1807.56 μM.

### 1-2: Confirmation of Anti-Cancer Effect of Combination Treatment

Based on Table 1 above and GI₅₀ values, omeprazole (KN510) and/or thioridazine (KN714) concentrations were added to each well such that the concentrations were as shown in the following groups, and then cancer cell death was measured in the same manner as in Examples 1-1 above.
1) Control,
2) Thioridazine (KN714) 5 μM alone treatment group,
3) Thioridazine (KN714) 10 μM alone treatment group,
4) Omeprazole (KN510) 200 μM alone treatment group,
5) Omeprazole (KN510) 200 μM+thioridazine (KN714) 5 μM combination treatment group,
6) Omeprazole (KN510) 200 μM+thioridazine (KN714) 10 μM combination treatment group.

As a comparative example, omeprazole (KN510) and/or trimetazidine (KN713) concentrations were added to each well such that the concentrations were as shown in the following groups, and then cancer cell killing was measured in the same manner as in Examples 1-1 above.
1) Control,
2) Omeprazole (KN510) 100 μM alone treatment group,
3) Omeprazole (KN510) 200 μM alone treatment group,
4) Trimetazidine (KN713) 2.5 mM alone treatment group,
5) Omeprazole (KN510) 100 μM+trimetazidine (KN713) 2.5 mM combination treatment group,
6) Omeprazole (KN510) 200 μM+trimetazidine (KN713) 2.5 mM combination treatment group.

TABLE 2
Cancer cell growth rate in response to omeprazole (KN510)
and thioridazine (KN714) combination treatment

| Conc. | Average (%) | STD (±) |
|---|---|---|
| Control | 100.00 | 0.68 |
| KN714 5 μM | 68.62 | 2.01 |
| KN714 10 μM | −5.25 | 0.67 |
| KN510 200 μM | 25.89 | 2.56 |
| KN714 5 μM + KN510 200 μM | 8.71 | 1.94 |
| KN714 10 μM + KN510 200 μM | −20.90 | 0.66 |

TABLE 3
Cancer cell growth rate following combination treatment
with omeprazole (KN510) and trimetazidine (KN713)

| Conc. | Average (%) | STD (±) |
|---|---|---|
| Control | 100.00 | 4.04 |
| KN510 100 μM | 69.73 | 10.56 |
| KN510 200 μM | 35.32 | 8.31 |
| KN713 2.5 mM | 88.90 | 10.08 |
| KN510 100 μM + KN713 2.5 mM | 52.79 | 13.70 |
| KN510 200 μM + KN713 2.5 mM | −3.84 | 0.47 |

Treatment of a pancreatic cancer cell line (MIA PaCa-2) with omeprazole (KN510) and thioridazine (KN714), individually or in combination, resulted in a significant increase in cancer cell killing effect when omeprazole (KN510) and thioridazine (KN714) were administered in combination compared to either treatment alone, as shown in FIG. 2A and Table 2.

Furthermore, we found that the combination of omeprazole (KN510) and thioridazine (KN714) of the present invention was superior in killing cancer cells compared to the combination of omeprazole (KN510) and trimetazidine (KN713), a 3-ketoacyl CoA thiolase inhibitor (FIG. 2B and Table 3).

### 1-3: Confirmation of Synergistic Effects of Combination Treatment

When the anticancer activity of two compounds in combination is greater than the simple sum of the anticancer activity of the individual compounds (expected activity), this is called synergistic. The predicted synergistic activity of a combination of the present invention can be calculated using the Colby Equation in Equation 1 (S.R. Colby, Weeds, 1967, pp. 15, 20-22):

$$E = α + β - ( α \times β \div 100 ) \qquad [ Equation 1 ]$$

α and β are the measured anti-cancer activity of each compound alone, and E is the predicted anti-cancer activity of a mixture of α and β as a prediction.

In the present invention, the actual measure of anti-cancer activity of the combination treatment was the inhibition rate of cancer cell growth (%), and the predicted value (E) of the combination treatment was measured by substituting it into the above Colby Equation. If the actual value is greater than the predicted value, it can be judged that there is a synergistic effect, and in this invention, the predicted value calculated by the Colby Equation and the actual value were compared to further verify the synergistic effect of the combination.

TABLE 4
Synergistic anti-cancer effects of combination treatment

| | Inhibition rate of | |
|---|---|---|
| | cancer cell growth (%) | Predicted value |
| Conc. | (Actual value) | (%) |
| Control | 0 | |
| KN714 5 μM | 31.38 | |
| KN714 10 μM | 105.25 | |
| KN510 200 μM | 74.11 | |
| KN714 5 μM | 91.29 | 82.24 |
| KN510 200 μM | | |
| KN714 10 μM | 120.90 | 101.36 |
| KN510 200 μM | | |

As a result, as shown in Table 4 above, the inhibition rate (%) of cancer cell growth by the combination of omeprazole (KN510) and thioridazine (KN714) was higher than the predicted value (%). In other words, it was found that omeprazole (KN510) and thioridazine (KN714) have a synergistic anticancer effect by combination treatment.

### Example 2: Confirmation of Anti-Cancer Activity Against Breast Cancer by Co-Administration of Omeprazole (KN510) and Thioridazine (KN714)

Experiments were performed using the breast cancer cell lines MCF-7 cells (1.2×10⁴ cells/well) and MDA-MB-231 cells (8.0×10³ cells/well) in the same manner as in Examples 1-2 and 1-3 above.

TABLE 5
Breast cancer cell growth rate in response to combination treatment

| | MCF-7 | | MDA-MB-231 | |
|---|---|---|---|---|
| | | Standard | | Standard |
| | Average | deviation | Average | deviation |
| Breast cancer | (%) | (±) | (%) | (±) |
| Control | 100.00 | 0.61 | 100.00 | 1.92 |
| KN510 100 μM | 65.30 | 1.06 | 70.29 | 12.22 |
| KN510 200 μM | 49.96 | 0.91 | 28.13 | 6.60 |
| KN714 5 μM | 98.51 | 0.87 | 90.64 | 6.30 |
| KN714 10 μM | 39.69 | 3.14 | 70.20 | 11.12 |
| KN510 100 μM + | 42.53 | 2.59 | 60.07 | 10.35 |
| KN714 5 μM | | | | |
| KN510 100 μM + | 21.83 | 1.94 | 44.43 | 2.23 |
| KN714 10 μM | | | | |
| KN510 200 μM + | 13.08 | 0.55 | 19.35 | 3.35 |
| KN714 5 μM | | | | |
| KN510 200 μM + | −2.37 | 2.41 | 9.38 | 4.37 |
| KN714 10 μM | | | | |

As a result, as shown in FIG. 3 and Table 5, co-treatment of breast cancer cells with omeprazole (KN510) and thioridazine (KN714) significantly inhibited breast cancer cell growth compared to the single treatment group.

TABLE 6
Synergistic anti-cancer effects on breast
cancer cells by combination treatment

| | MCF-7 | | MDA-MB-231 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Breast cancer | value) | value (%) | value) | value (%) |
| Control | 0.00 | | 0.00 | |
| KN510 100 μM | 34.70 | | 29.71 | |
| KN510 200 μM | 50.04 | | 71.87 | |
| KN714 5 μM | 1.49 | | 9.36 | |
| KN714 10 μM | 60.31 | | 29.80 | |
| KN510 100 μM + | 57.47 | 35.67 | 39.93 | 36.29 |
| KN714 5 μM | | | | |
| KN510 100 μM + | 78.17 | 74.08 | 55.57 | 50.66 |
| KN714 10 μM | | | | |
| KN510 200 μM + | 86.92 | 50.78 | 80.65 | 74.50 |
| KN714 5 μM | | | | |
| KN510 200 μM + | 102.37 | 80.17 | 90.62 | 80.25 |
| KN714 10 μM | | | | |

As shown in Table 6, the synergistic effects of the combination of omeprazole (KN510) and thioridazine (KN714) in breast cancer cells were confirmed.

### Example 3: Confirmation of Anti-Cancer Activity Against Colon Cancer by Co-Administration of Omeprazole (KN510) and Thioridazine (KN714)

Experiments were performed using the colon cancer cell lines DLD-1 cells (8.0×10³ cells/well) and HCT-116 cells (8.0×10³ cells/well) in the same manner as in Examples 1-2 and 1-3 above.

TABLE 7
Colon cancer cell growth rate in response to combination treatment

| | DLD-1 | | HCT-116 | |
|---|---|---|---|---|
| | | Standard | | Standard |
| | Average | deviation | Average | deviation |
| Colon cancer | (%) | (±) | (%) | (±) |
| Control | 100.00 | 4.58 | 100.00 | 0.81 |
| KN510 100 μM | 73.90 | 4.09 | 84.28 | 1.13 |
| KN510 200 μM | 36.78 | 0.17 | 68.86 | 2.05 |
| KN714 5 μM | 91.34 | 3.62 | 92.01 | 0.67 |
| KN714 10 μM | 86.87 | 1.13 | 67.80 | 0.93 |
| KN510 100 μM + | 56.55 | 2.63 | 61.45 | 1.18 |
| KN714 5 μM | | | | |
| KN510 100 μM + | 39.67 | 2.92 | 46.92 | 1.89 |
| KN714 10 μM | | | | |
| KN510 200 μM + | 28.09 | 0.42 | 37.73 | 0.96 |
| KN714 5 μM | | | | |
| KN510 200 μM + | 15.88 | 0.23 | 20.99 | 1.33 |
| KN714 10 μM | | | | |

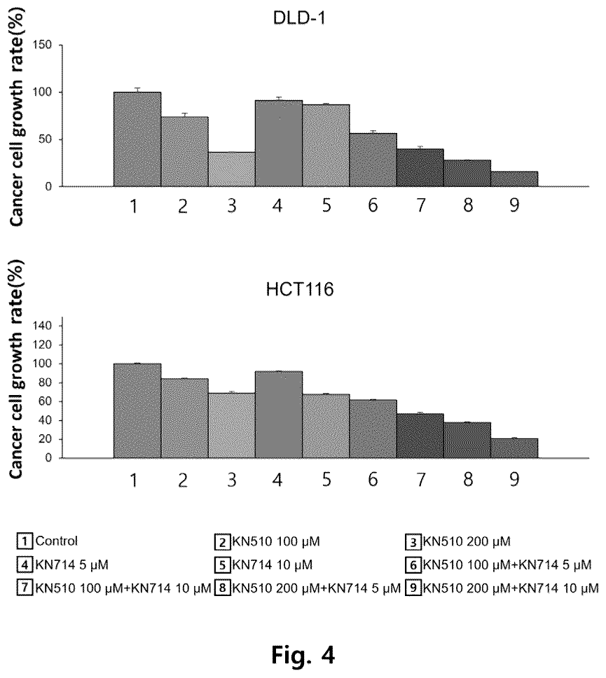

As a result, as shown in FIG. 4 and Table 7, we found that co-treatment of colon cancer cells with omeprazole (KN510) and thioridazine (KN714) significantly inhibited colon cancer cell growth compared to the single treatment group.

TABLE 8
Synergistic anti-cancer effects on colon
cancer cells by combination treatment

| | DLD-1 | | HCT-116 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Colon cancer | value) | value (%) | value) | value (%) |
| Control | 0.00 | | 0.00 | |
| KN510 100 μM | 26.10 | | 15.72 | |
| KN510 200 μM | 63.22 | | 31.14 | |
| KN714 5 μM | 8.66 | | 7.99 | |
| KN714 10 μM | 13.13 | | 32.20 | |
| KN510 100 μM + | 43.45 | 32.50 | 38.55 | 22.45 |
| KN714 5 μM | | | | |
| KN510 100 μM + | 60.33 | 35.80 | 53.08 | 42.85 |
| KN714 10 μM | | | | |
| KN510 200 μM + | 71.91 | 66.40 | 62.27 | 36.64 |
| KN714 5 μM | | | | |
| KN510 200 μM + | 84.12 | 68.05 | 79.01 | 53.31 |
| KN714 10 μM | | | | |

As shown in Table 8, the synergistic effects of the combination of omeprazole (KN510) and thioridazine (KN714) in colon cancer cells were confirmed.

### Example 4: Confirmation of Anti-Cancer Activity Against Glioblastoma by Co-Administration of Omeprazole (KN510) and Thioridazine (KN714)

Experiments were performed using the glioblastoma (GBM) cell lines SF295 cells (8.0×10³ cells/well) and U251 cells (8.0×10³ cells/well) in the same manner as in Examples 1-2 and 1-3 above.

TABLE 9
Glioblastoma cell growth rate in response to combination treatment

| | SF295 | | U251 | |
|---|---|---|---|---|
| | | Standard | | Standard |
| | Average | deviation | Average | deviation |
| Glioblastoma | (%) | (±) | (%) | (±) |
| Control | 100.00 | 2.59 | 100.00 | 9.26 |
| KN510 100 μM | 67.98 | 3.24 | 73.51 | 5.79 |
| KN510 200 μM | 36.53 | 0.89 | 55.11 | 6.99 |
| KN714 5 μM | 83.54 | 0.66 | 59.99 | 4.76 |
| KN714 10 μM | 24.47 | 1.44 | −20.54 | 0.77 |
| KN510 100 μM + | 35.39 | 1.86 | 8.51 | 0.47 |
| KN714 5 μM | | | | |
| KN510 100 μM + | 7.53 | 1.66 | −30.07 | 0.88 |
| KN714 10 μM | | | | |
| KN510 200 μM + | 12.83 | 2.74 | −4.00 | 2.08 |
| KN714 5 μM | | | | |
| KN510 200 μM + | −2.64 | 0.79 | −27.47 | 0.44 |
| KN714 10 μM | | | | |

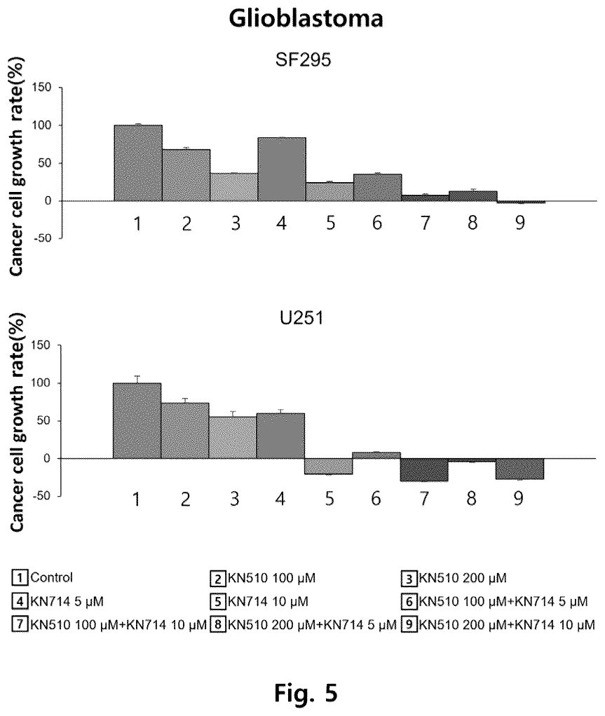

As a result, as shown in FIG. 5 and Table 9, co-treatment of glioblastoma cells with omeprazole (KN510) and thioridazine (KN714) significantly inhibited glioblastoma cell growth compared to the single treatment group.

TABLE 10
Synergistic anti-cancer effects on glioblastoma
cells by combination treatment

| | SF295 | | U251 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Glioblastoma | value) | value (%) | value) | value (%) |
| Control | 0.00 | | 0.00 | |
| KN510 100 μM | 32.02 | | 26.49 | |
| KN510 200 μM | 63.47 | | 44.89 | |
| KN714 5 μM | 16.46 | | 40.01 | |
| KN714 10 μM | 75.53 | | 120.54 | |
| KN510 100 μM + | 64.61 | 43.21 | 91.49 | 55.90 |
| KN714 5 μM | | | | |
| KN510 100 μM + | 92.47 | 83.37 | 130.07 | 115.10 |
| KN714 10 μM | | | | |
| KN510 200 μM + | 87.17 | 69.49 | 104.00 | 66.94 |
| KN714 5 μM | | | | |
| KN510 200 μM + | 102.64 | 91.06 | 127.47 | 111.32 |
| KN714 10 μM | | | | |

As shown in Table 10, the synergistic effects of the combination of omeprazole (KN510) and thioridazine (KN714) in glioblastoma cells were confirmed.

### Example 5: Confirmation of Anticancer Activity Against Liver Cancer by Co-Administration of Omeprazole (KN510) and Thioridazine (KN714)

Experiments were performed using the liver cancer cell lines Hep-3B cells (1.5×10⁴ cells/well) and SK-HEP-1 cells (1.2×10⁴ cells/well) in the same manner as in Examples 1-2 and 1-3 above.

TABLE 11
Liver cancer cell growth rate in response to combination treatment

| | Hep-3B | | SK-HEP-1 | |
|---|---|---|---|---|
| | | Standard | | Standard |
| | Average | deviation | Average | deviation |
| Liver cancer | (%) | (±) | (%) | (±) |
| Control | 100.00 | 0.73 | 100.00 | 1.72 |
| KN510 100 μM | 84.95 | 2.72 | 53.28 | 1.06 |
| KN510 200 μM | 59.89 | 4.16 | 37.19 | 2.24 |
| KN714 5 μM | 93.94 | 0.78 | 82.37 | 0.90 |
| KN714 10 μM | 70.15 | 1.13 | 59.94 | 0.96 |
| KN510 100 μM + | 68.01 | 4.79 | 45.05 | 2.98 |
| KN714 5 μM | | | | |
| KN510 100 μM + | 30.77 | 0.84 | 13.59 | 1.77 |
| KN714 10 μM | | | | |
| KN510 200 μM + | 67.25 | 1.80 | 41.89 | 0.90 |
| KN714 5 μM | | | | |
| KN510 200 μM + | 21.81 | 2.31 | −1.57 | 1.04 |
| KN714 10 μM | | | | |

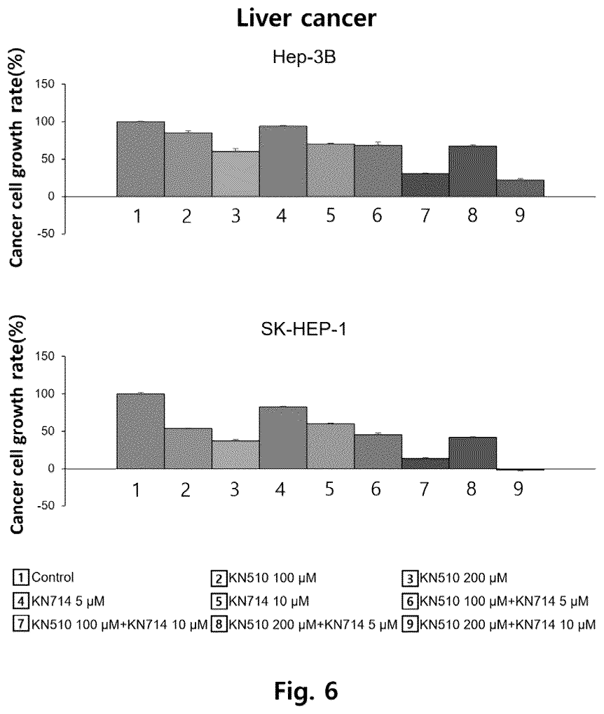

As a result, as shown in FIG. 6 and Table 11, the combination treatment of liver cancer cells with omeprazole (KN510) and thioridazine (KN714) significantly inhibited the growth of liver cancer cells compared to the single treatment group.

TABLE 12
Synergistic anti-cancer effects on liver
cancer cells by combination treatment

| | Hep-3B | | SK-HEP-1 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Liver cancer | value) | value (%) | value) | value (%) |
| Control | 0.00 | | 0.00 | |
| KN510 100 μM | 15.05 | | 46.72 | |
| KN510 200 μM | 40.11 | | 62.81 | |
| KN714 5 μM | 6.06 | | 17.63 | |
| KN714 10 μM | 29.85 | | 40.06 | |
| KN510 100 μM + | 31.99 | 20.20 | 54.95 | 56.11 |
| KN714 5 μM | | | | |
| KN510 100 μM + | 69.23 | 40.41 | 86.41 | 68.06 |
| KN714 10 μM | | | | |
| KN510 200 μM + | 32.75 | 43.74 | 58.11 | 69.37 |
| KN714 5 μM | | | | |
| KN510 200 μM + | 78.19 | 57.99 | 101.57 | 77.71 |
| KN714 10 μM | | | | |

As shown in Table 12, the synergistic effects of omeprazole (KN510) and thioridazine (KN714) in combination in liver cancer cells were confirmed.

### Example 6: Confirmation of Anti-Leukemic Activity of Omeprazole (KN510) and Thioridazine (KN714) in Combination Against Leukemia

Experiments were performed using the leukemia cell lines K562 cells (1.0×10⁴ cells/well) and SR cells (2.0×10⁴ cells/well) in the same manner as in Examples 1-2 and 1-3 above.

TABLE 13
Leukemia cell growth rate in response to combination treatment

| | K562 | | SR | |
|---|---|---|---|---|
| | | Standard | | Standard |
| | Average | deviation | Average | deviation |
| Leukemia | (%) | (±) | (%) | (±) |
| Control | 100.00 | 2.72 | 100.00 | 6.77 |
| KN510 100 μM | 55.18 | 0.77 | 64.90 | 5.59 |
| KN510 200 μM | 27.01 | 0.65 | 29.89 | 6.17 |
| KN714 5 μM | 42.25 | 2.98 | 62.75 | 4.28 |
| KN714 10 μM | −1.56 | 0.55 | −11.34 | 3.15 |
| KN510 100 μM + | 16.74 | 2.51 | 33.02 | 4.92 |
| KN714 5 μM | | | | |
| KN510 100 μM + | −1.78 | 0.77 | −7.47 | 2.42 |
| KN714 10 μM | | | | |
| KN510 200 μM + | 6.56 | 1.38 | 0.09 | 7.55 |
| KN714 5 μM | | | | |
| KN510 200 μM + | −3.49 | 1.88 | −14.78 | 1.45 |
| KN714 10 μM | | | | |

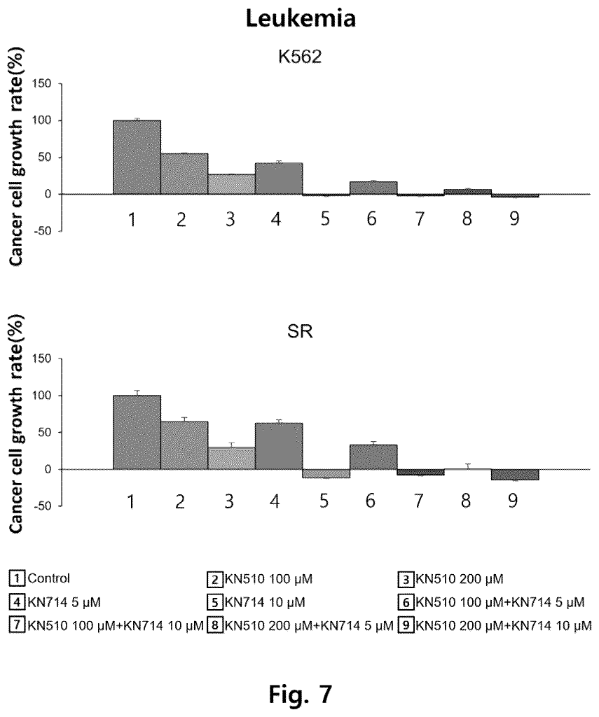

As a result, as shown in FIG. 7 and Table 13, the combination treatment of leukemia cells with omeprazole (KN510) and thioridazine (KN714) significantly inhibited leukemia cell growth compared to the single treatment group.

TABLE 14
Synergistic anti-cancer effects on leukemia
cells by combination treatment

| | K562 | | SR | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Leukemia | value) | value (%) | value) | value (%) |
| Control | 0.00 | | 0.00 | |
| KN510 100 μM | 44.82 | | 35.10 | |
| KN510 200 μM | 72.99 | | 70.11 | |
| KN714 5 μM | 57.75 | | 37.25 | |
| KN714 10 μM | 101.56 | | 111.34 | |
| KN510 100 μM + | 83.26 | 76.69 | 66.98 | 59.28 |
| KN714 5 μM | | | | |
| KN510 100 μM + | 101.78 | 100.86 | 107.47 | 107.36 |
| KN714 10 μM | | | | |
| KN510 200 μM + | 93.44 | 88.59 | 99.91 | 81.24 |
| KN714 5 μM | | | | |
| KN510 200 μM + | 103.49 | 100.42 | 114.78 | 103.39 |
| KN714 10 μM | | | | |

As shown in Table 14, the synergistic effects of the combination of omeprazole (KN510) and thioridazine (KN714) in leukemia cells were confirmed.

### Example 7: Confirmation of Anticancer Activity Against Melanoma by Co-Administration of Omeprazole (KN510) and Thioridazine (KN714)

Experiments were performed using the melanoma cell lines UACC-62 cells (1.0×10⁴ cells/well) and SK-MEL-5 (1.0×10⁴ cells/well) in the same manner as in Examples 1-2 and 1-3 above.

TABLE 15
Melanoma cell growth rate in response to combination treatment

| | UACC-62 | | SK-MEL-5 | |
|---|---|---|---|---|
| | | Standard | | Standard |
| | Average | deviation | Average | deviation |
| Melanoma | (%) | (±) | (%) | (±) |
| Control | 100.00 | 2.16 | 100.00 | 1.79 |
| KN510 100 μM | 46.56 | 2.14 | 65.42 | 2.89 |
| KN510 200 μM | 3.59 | 1.55 | 46.11 | 6.22 |
| KN714 5 μM | 89.43 | 8.95 | 80.69 | 8.16 |
| KN714 10 μM | 39.54 | 3.77 | −23.56 | 0.60 |
| KN510 100 μM + | 23.16 | 2.14 | 21.98 | 2.29 |
| KN714 5 μM | | | | |
| KN510 100 μM + | −29.49 | 1.32 | −29.43 | 2.16 |
| KN714 10 μM | | | | |
| KN510 200 μM + | 21.85 | 4.36 | 17.54 | 2.52 |
| KN714 5 μM | | | | |
| KN510 200 μM + | −43.00 | 3.09 | −40.13 | 2.57 |
| KN714 10 μM | | | | |

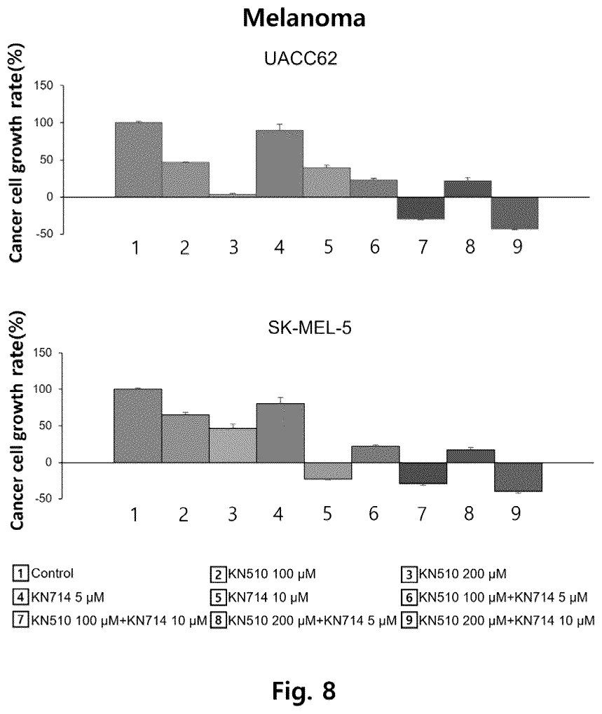

As a result, as shown in FIG. 8 and Table 15, we found that co-treatment of melanoma cells with omeprazole (KN510) and thioridazine (KN714) significantly inhibited melanoma cell growth compared to the single treatment group.

TABLE 16
Synergistic anti-cancer effects on melanoma
cells by combination treatment

| | UACC-62 | | SK-MEL-5 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Melanoma | value) | value (%) | value) | value (%) |
| Control | 0.00 | | 0.00 | |
| KN510 100 μM | 53.44 | | 34.58 | |
| KN510 200 μM | 96.41 | | 53.89 | |
| KN714 5 μM | 10.57 | | 19.31 | |
| KN714 10 μM | 60.46 | | 123.56 | |
| KN510 100 μM + | 76.84 | 58.36 | 78.02 | 47.21 |
| KN714 5 μM | | | | |
| KN510 100 μM + | 129.49 | 81.59 | 129.43 | 115.41 |
| KN714 10 μM | | | | |
| KN510 200 μM + | 78.15 | 96.79 | 82.46 | 62.79 |
| KN714 5 μM | | | | |
| KN510 200 μM + | 143.00 | 98.58 | 140.13 | 110.86 |
| KN714 10 μM | | | | |

As shown in Table 16, the synergistic effects of the combination of omeprazole (KN510) and thioridazine (KN714) in melanoma cells were confirmed.

### Example 8: Confirmation of Anti-Cancer Activity Against Lung Cancer by Co-Administration of Omeprazole (KN510) and Thioridazine (KN714)

Experiments were performed in the same manner as in Examples 1-2 and 1-3 above using lung cancer cells, A549 cells (1.0×10⁴ cells/well) and H1975 cells (1.0×10⁴ cells/well).

TABLE 17
Lung cancer cell growth rate in response to combination treatment

| | A549 | | H1975 | |
|---|---|---|---|---|
| | | Standard | | Standard |
| | Average | deviation | Average | deviation |
| Lung cancer | (%) | (±) | (%) | (±) |
| Control | 100.00 | 2.42 | 100.00 | 1.12 |
| KN510 100 μM | 55.44 | 3.52 | 84.54 | 2.09 |
| KN510 200 μM | 30.29 | 3.17 | 43.18 | 5.73 |
| KN714 5 μM | 97.85 | 1.18 | 113.89 | 2.27 |
| KN714 10 μM | 64.57 | 3.29 | 91.90 | 2.35 |
| KN510 100 μM + | 40.80 | 1.78 | 63.83 | 2.17 |
| KN714 5 μM | | | | |
| KN510 100 μM + | 20.76 | 2.76 | 34.72 | 0.82 |
| KN714 10 μM | | | | |
| KN510 200 μM + | 19.21 | 1.41 | 21.96 | 1.83 |
| KN714 5 μM | | | | |
| KN510 200 μM + | 5.80 | 2.13 | 11.10 | 1.40 |
| KN714 10 μM | | | | |

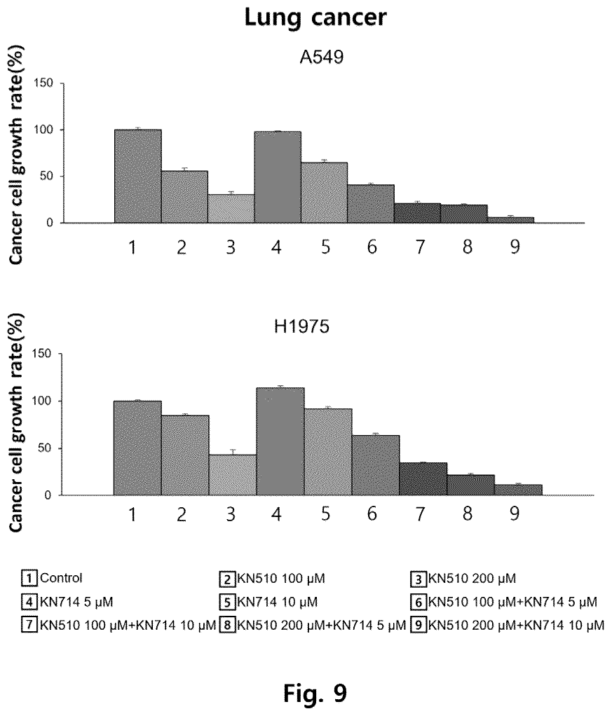

As a result, as shown in FIG. 9 and Table 17, the combination treatment of lung cancer cells with omeprazole (KN510) and thioridazine (KN714) significantly inhibited lung cancer cell growth compared to the single treatment group.

TABLE 18
Synergistic anti-cancer effects on lung
cancer cells by combination treatment

| | A549 | | H1975 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Lung cancer | value) | value (%) | value) | value (%) |
| Control | 0.00 | | 0.00 | |
| KN510 100 μM | 44.56 | | 15.46 | |
| KN510 200 μM | 69.71 | | 56.82 | |
| KN714 5 μM | 2.15 | | −13.89 | |
| KN714 10 μM | 35.43 | | 8.10 | |
| KN510 100 μM + | 59.20 | 45.75 | 36.17 | 3.72 |
| KN714 5 μM | | | | |
| KN510 100 μM + | 79.24 | 64.20 | 65.28 | 22.31 |
| KN714 10 μM | | | | |
| KN510 200 μM + | 80.79 | 70.36 | 78.04 | 50.82 |
| KN714 5 μM | | | | |
| KN510 200 μM + | 94.20 | 80.44 | 88.90 | 60.32 |
| KN714 10 μM | | | | |

As shown in Table 18, the synergistic effects of omeprazole (KN510) and thioridazine (KN714) in combination in lung cancer cells were confirmed.

### Example 9: Confirmation of Anti-Cancer Activity Against Ovarian Cancer by Co-Administration of Omeprazole (KN510) and Thioridazine (KN714)

Experiments were performed using ovarian cancer cells, OVCAR-8 cells (1.0×10⁴ cells/well) and SK-OV-3 cells (1.0×10⁴ cells/well) in the same manner as in Examples 1-2 and 1-3 above.

TABLE 19
Ovarian cancer cell growth rate in
response to combination treatment

| | OVCAR-8 | | SK-OV-3 | |
|---|---|---|---|---|
| | | Standard | | Standard |
| | Average | deviation | Average | deviation |
| Ovarian cancer | (%) | (±) | (%) | (±) |
| Control | 100.00 | 5.18 | 100.00 | 5.59 |
| KN510 100 μM | 55.49 | 2.71 | 88.53 | 8.72 |
| KN510 200 μM | 36.66 | 5.07 | 40.18 | 11.93 |
| KN714 5 μM | 93.08 | 3.44 | 92.18 | 6.25 |
| KN714 10 μM | 44.89 | 2.79 | 62.36 | 2.48 |
| KN510 100 μM + | 23.46 | 2.22 | 66.35 | 3.56 |
| KN714 5 μM | | | | |
| KN510 100 μM + | 12.86 | 0.76 | 14.08 | 5.84 |
| KN714 10 μM | | | | |
| KN510 200 μM + | 7.32 | 1.72 | 28.06 | 4.49 |
| KN714 5 μM | | | | |
| KN510 200 μM + | −3.38 | 0.82 | −16.47 | 2.21 |
| KN714 10 μM | | | | |

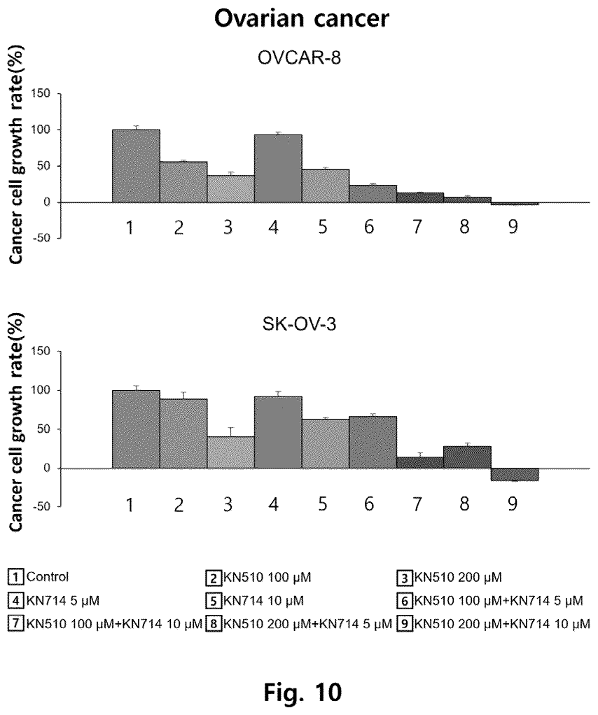

As a result, as shown in FIG. 10 and Table 19, the combination treatment of ovarian cancer cells with omeprazole (KN510) and thioridazine (KN714) significantly inhibited the growth of ovarian cancer cells compared to the single treatment group.

TABLE 20
Synergistic anti-cancer effects on ovarian
cancer cells by combination treatment

| | OVCAR-8 | | SK-OV-3 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Ovarian cancer | value) | value (%) | value) | value (%) |
| Control | 0.00 | | 0.00 | |
| KN510 100 μM | 44.51 | | 11.47 | |
| KN510 200 μM | 63.34 | | 59.82 | |
| KN714 5 μM | 6.92 | | 7.82 | |
| KN714 10 μM | 55.11 | | 37.64 | |
| KN510 100 μM + | 76.54 | 48.35 | 33.65 | 18.39 |
| KN714 5 μM | | | | |
| KN510 100 μM + | 87.14 | 75.09 | 85.92 | 44.79 |
| KN714 10 μM | | | | |
| KN510 200 μM + | 92.68 | 65.87 | 71.94 | 62.96 |
| KN714 5 μM | | | | |
| KN510 200 μM + | 103.38 | 83.54 | 116.47 | 74.94 |
| KN714 10 μM | | | | |

As shown in Table 20 above, the synergistic effect of omeprazole (KN510) and thioridazine (KN714) in ovarian cancer cells were confirmed.

### Example 10: Confirmation of Anti-Cancer Activity Against Prostate Cancer by Co-Administration of Omeprazole (KN510) and Thioridazine (KN714)

Experiments were performed using prostate cancer cells, PC-3 cells (8.0×10³ cells/well) and DU-145 cells (1.0×10⁴ cells/well), in the same manner as in Examples 1-2 and 1-3 above.

TABLE 21
Prostate cancer cell growth rate in
response to combination treatment

| | PC-3 | | DU-145 | |
|---|---|---|---|---|
| | | Standard | | Standard |
| | Average | deviation | Average | deviation |
| Prostate cancer | (%) | (±) | (%) | (±) |
| Control | 100.00 | 2.41 | 100.00 | 1.80 |
| KN510 100 μM | 53.42 | 2.56 | 49.59 | 2.67 |
| KN510 200 μM | 33.81 | 2.99 | 37.08 | 1.35 |
| KN714 5 μM | 81.94 | 1.99 | 75.49 | 3.02 |
| KN714 10 μM | 47.00 | 1.98 | 55.27 | 2.35 |
| KN510 100 μM + | 28.74 | 7.98 | 37.64 | 0.80 |
| KN714 5 μM | | | | |
| KN510 100 μM + | 7.55 | 0.61 | 10.85 | 0.42 |
| KN714 10 μM | | | | |
| KN510 200 μM + | 31.14 | 2.37 | 35.88 | 1.18 |
| KN714 5 μM | | | | |
| KN510 200 μM + | −7.66 | 0.97 | −8.37 | 2.17 |
| KN714 10 μM | | | | |

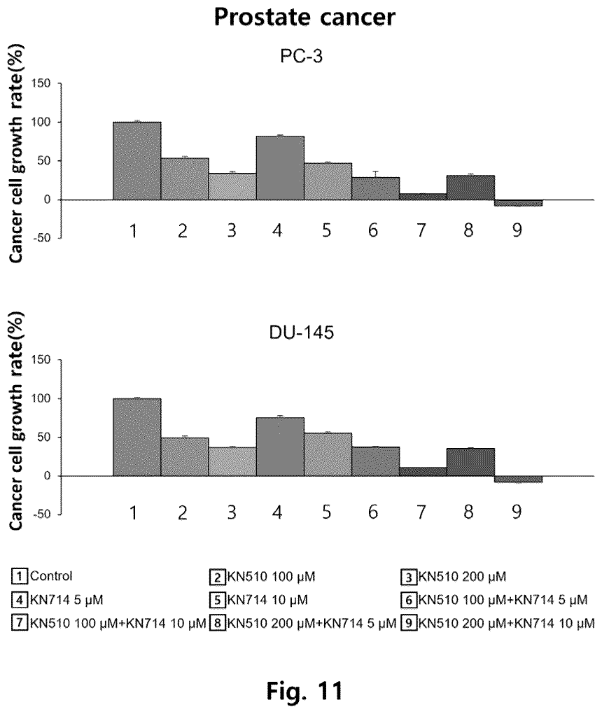

As a result, as shown in FIG. 11 and Table 21, the combination treatment of prostate cancer cells with omeprazole (KN510) and thioridazine (KN714) significantly inhibited prostate cancer cell growth compared to the single treatment group.

TABLE 22
Synergistic anti-cancer effects on prostate
cancer cells by combination treatment

| | PC-3 | | DU-145 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Prostate cancer | value) | value (%) | value) | value (%) |
| Control | 0.00 | | 0.00 | |
| KN510 100 μM | 46.58 | | 50.41 | |
| KN510 200 μM | 66.19 | | 62.92 | |
| KN714 5 μM | 18.06 | | 24.51 | |
| KN714 10 μM | 53.00 | | 44.73 | |
| KN510 100 μM + | 71.26 | 56.23 | 62.36 | 62.56 |
| KN714 5 μM | | | | |
| KN510 100 μM + | 92.45 | 74.89 | 89.15 | 72.59 |
| KN714 10 μM | | | | |
| KN510 200 μM + | 68.86 | 72.29 | 64.12 | 72.01 |
| KN714 5 μM | | | | |
| KN510 200 μM + | 107.66 | 84.11 | 108.37 | 79.51 |
| KN714 10 μM | | | | |

As shown in Table 22 above, the synergistic effects of omeprazole (KN510) and thioridazine (KN714) in combination in prostate cancer cells were confirmed.

### Example 11: Confirmation of Anti-Cancer Activity Against Pancreatic Cancer by Co-Administration of Omeprazole (KN510) and Thioridazine (KN714)

Experiments were performed using the pancreatic cancer cell lines MIA PaCa-2 cells (1.2×10⁴ cells/well) and PANC-1 cells (1.2×10⁴ cells/well) in the same manner as in Examples 1-2 and 1-3 above.

TABLE 23
Pancreatic cancer cell growth rate in
response to combination treatment

| | MIA PaCa-2 | | PANC-1 | |
|---|---|---|---|---|
| | | Standard | | Standard |
| | Average | deviation | Average | deviation |
| Pancreatic cancer | (%) | (±) | (%) | (±) |
| Control | 100.00 | 1.50 | 100.00 | 4.36 |
| KN510 100 μM | 69.78 | 0.81 | 44.47 | 3.06 |
| KN510 200 μM | 21.75 | 2.62 | 10.96 | 1.34 |
| KN714 5 μM | 76.62 | 3.77 | 89.85 | 5.52 |
| KN714 10 μM | −5.93 | 0.97 | 30.04 | 8.40 |
| KN510 100 μM + | 25.34 | 7.47 | 4.28 | 3.94 |
| KN714 5 μM | | | | |
| KN510 100 μM + | −20.92 | 2.71 | −5.08 | 3.94 |
| KN714 10 μM | | | | |
| KN510 200 μM + | −16.71 | 1.25 | −7.70 | 2.47 |
| KN714 5 μM | | | | |
| KN510 200 μM + | −32.38 | 0.98 | −20.74 | 2.65 |
| KN714 10 μM | | | | |

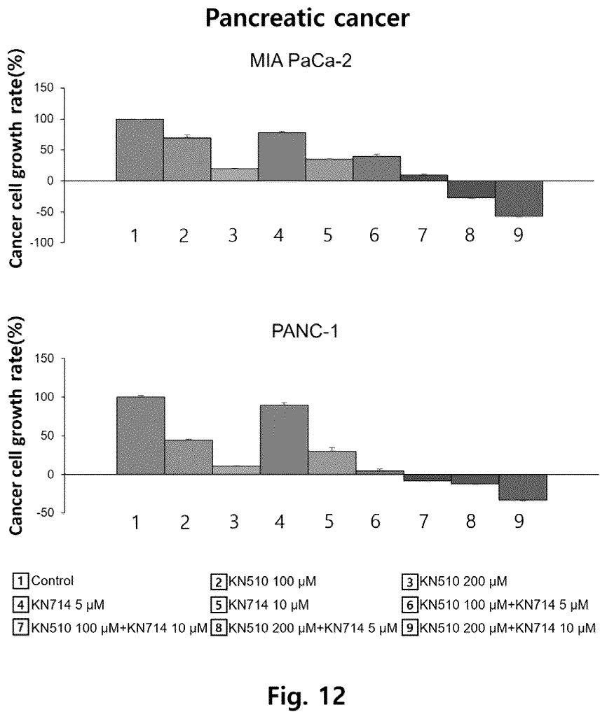

As a result, as shown in FIG. 12 and Table 23, the combination treatment of pancreatic cancer cells with omeprazole (KN510) and thioridazine (KN714) significantly inhibited pancreatic cancer cell growth compared to the single treatment group.

TABLE 24
Synergistic anti-cancer effects on pancreatic
cancer cells by combination treatment

| | MIA PaCa-2 | | PANC-1 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Pancreatic cancer | value) | value (%) | value) | value (%) |
| Control | 0.00 | | 0.00 | |
| KN510 100 μM | 30.22 | | 55.53 | |
| KN510 200 μM | 78.25 | | 89.04 | |
| KN714 5 μM | 23.38 | | 10.15 | |
| KN714 10 μM | 105.93 | | 69.96 | |
| KN510 100 μM + | 74.66 | 46.54 | 95.72 | 60.05 |
| KN714 5 μM | | | | |
| KN510 100 μM + | 120.92 | 104.14 | 105.08 | 86.64 |
| KN714 10 μM | | | | |
| KN510 200 μM + | 116.71 | 83.34 | 107.70 | 90.16 |
| KN714 5 μM | | | | |
| KN510 200 μM + | 132.38 | 101.29 | 120.74 | 96.71 |
| KN714 10 μM | | | | |

As shown in Table 24, the synergistic effects of omeprazole (KN510) and thioridazine (KN714) in combination in pancreatic cancer cells were confirmed.

### Example 12: Confirmation of anti-cancer activity against renal cell carcinoma by co-administration of omeprazole (KN510) and thioridazine (KN714)

Experiments were performed using the Renal cell carcinoma cell lines ACHN cells (1.0×10⁴ cells/well) and CAKI-1 cells (8.0×10³ cells/well) in the same manner as in Examples 1-2 and 1-3 above.

TABLE 25
renal cell carcinoma cell growth rate
in response to combination treatment

| | ACHN | | CAKI-1 | |
|---|---|---|---|---|
| | | Standard | | Standard |
| Renal cell | Average | deviation | Average | deviation |
| carcinoma | (%) | (±) | (%) | (±) |
| Control | 100.00 | 2.27 | 100.00 | 10.01 |
| KN510 100 μM | 61.12 | 1.98 | 67.99 | 13.62 |
| KN510 200 μM | 19.93 | 1.53 | 27.11 | 6.57 |
| KN714 5 μM | 102.76 | 0.56 | 75.99 | 5.26 |
| KN714 10 μM | 70.39 | 2.13 | 50.59 | 11.85 |
| KN510 100 μM + | 37.85 | 1.41 | 33.34 | 8.16 |
| KN714 5 μM | | | | |
| KN510 100 μM + | 21.32 | 0.74 | 25.74 | 3.66 |
| KN714 10 μM | | | | |
| KN510 200 μM + | 18.83 | 1.06 | 18.37 | 1.77 |
| KN714 5 μM | | | | |
| KN510 200 μM + | 4.72 | 0.80 | 11.29 | 2.51 |
| KN714 10 μM | | | | |

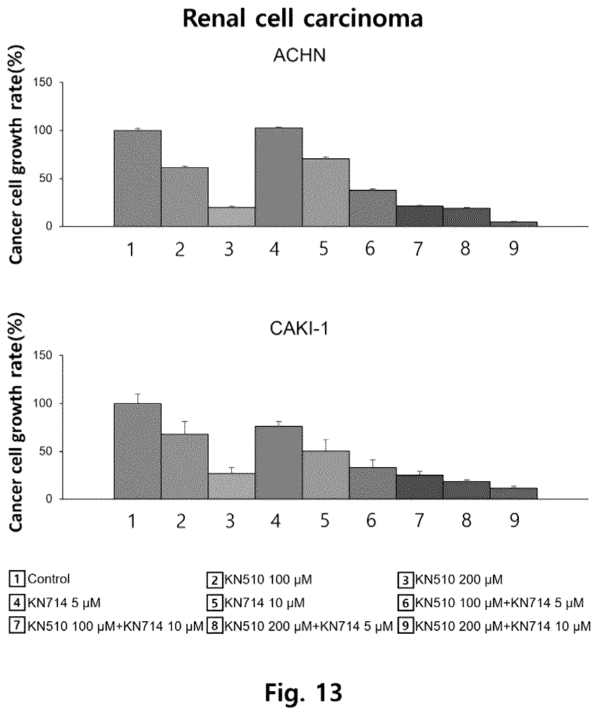

As a result, as shown in FIG. 13 and Table 25, we found that co-treatment of renal cell carcinoma cells with omeprazole (KN510) and thioridazine (KN714) significantly inhibited renal cell carcinoma cell growth compared to the single treatment group.

TABLE 26
Synergistic anti-cancer effects on renal cell
carcinoma cells by combination treatment

| | ACHN | | CAKI-1 | |
|---|---|---|---|---|
| | Inhibition | | Inhibition | |
| | rate of | | rate of | |
| | cancer cell | | cancer cell | |
| | growth (%) | | growth (%) | |
| Renal cell | (Actual | Predicted | (Actual | Predicted |
| carcinoma | value) | value (%) | value) | value (%) |
| Control | 0.00 | | 0.00 | |
| KN510 100 μM | 38.88 | | 32.01 | |
| KN510 200 μM | 80.07 | | 72.89 | |
| KN714 5 μM | −2.76 | | 24.01 | |
| KN714 10 μM | 29.61 | | 49.41 | |
| KN510 100 μM + | 62.15 | 37.20 | 66.66 | 48.33 |
| KN714 5 μM | | | | |
| KN510 100 μM + | 78.68 | 56.98 | 74.26 | 65.60 |
| KN714 10 μM | | | | |
| KN510 200 μM + | 81.17 | 79.52 | 81.63 | 79.40 |
| KN714 5 μM | | | | |
| KN510 200 μM + | 95.28 | 85.97 | 88.71 | 86.29 |
| KN714 10 μM | | | | |

As shown in Table 26, the synergistic effects of the combination of omeprazole (KN510) and thioridazine (KN714) in renal cell carcinoma cells were confirmed.

### Example 13: Confirmation of Anticancer Activity Against Gastric Cancer by Co-Administration of Omeprazole (KN510) and Thioridazine (KN714)

Experiments were performed using the stomach cancer cell lines AGS cells (1.0×10⁴ cells/well) and MKN-28 cells (1.0×10⁴ cells/well) in the same manner as in Examples 1-1 and 1-2 above.

TABLE 27
Gastric cancer cell growth rate in
response to combination treatment

| | AGS | | MKN-28 | |
|---|---|---|---|---|
| | | Standard | | Standard |
| | Average | deviation | Average | deviation |
| Stomach cancer | (%) | (±) | (%) | (±) |
| Control | 100.00 | 1.70 | 100.00 | 7.57 |
| KN510 100 μM | 60.39 | 6.67 | 49.80 | 1.15 |
| KN510 200 μM | 30.51 | 4.76 | 25.20 | 0.91 |
| KN714 5 μM | 67.13 | 2.56 | 91.81 | 5.91 |
| KN714 10 μM | 15.46 | 1.27 | 36.04 | 0.92 |
| KN510 100 μM + | 32.95 | 0.91 | 23.44 | 3.81 |
| KN714 5 μM | | | | |
| KN510 100 μM + | −11.10 | 0.90 | 1.78 | 2.39 |
| KN714 10 μM | | | | |
| KN510 200 μM + | 15.24 | 2.09 | 9.17 | 1.09 |
| KN714 5 μM | | | | |
| KN510 200 μM + | −15.59 | 0.53 | −5.41 | 1.20 |
| KN714 10 μM | | | | |

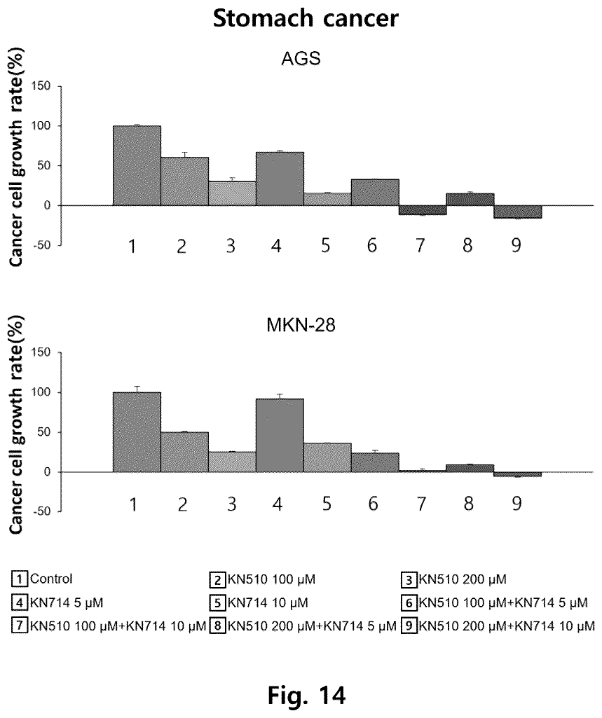

As a result, as shown in FIG. 14 and Table 27, we found that co-treatment of gastric cancer cells with omeprazole (KN510) and thioridazine (KN714) significantly inhibited gastric cancer cell growth compared to the single treatment group.

TABLE 28
Synergistic anti-cancer effects on gastric
cancer cells by combination treatment

| | AGS | | MKN-28 | |
|---|---|---|---|---|
| | Inhibition | | Inhibition | |
| | rate of | | rate of | |
| | cancer cell | | cancer cell | |
| | growth (%) | | growth (%) | |
| | (Actual | Predicted | (Actual | Predicted |
| Stomach cancer | value) | value (%) | value) | value (%) |
| Control | 0.00 | | 0.00 | |
| KN510 100 μM | 39.61 | | 50.20 | |
| KN510 200 μM | 69.49 | | 74.80 | |
| KN714 5 μM | 32.87 | | 8.19 | |
| KN714 10 μM | 84.54 | | 63.96 | |
| KN510 100 μM + | 67.05 | 59.46 | 76.56 | 54.28 |
| KN714 5 μM | | | | |
| KN510 100 μM + | 111.10 | 90.66 | 98.22 | 82.05 |
| KN714 10 μM | | | | |
| KN510 200 μM + | 84.76 | 79.51 | 90.83 | 76.86 |
| KN714 5 μM | | | | |
| KN510 200 μM + | 115.59 | 95.28 | 105.41 | 90.92 |
| KN714 10 μM | | | | |

As shown in Table 28, the synergistic effects of the combination of omeprazole (KN510) and thioridazine (KN714) on gastric cancer cells were confirmed.

The composition comprising the carnitine acylcarnitine carrier inhibitor and peroxisome beta-oxidation inhibitor of the present invention not only significantly reduced cancer cell growth compared to the use of each drug alone, but also confirmed the synergistic anti-cancer effect of co-administration, thus making them useful as effective combination anti-cancer agents.

1. A pharmaceutical composition for the prevention or treatment of cancer, comprising a carnitine acylcarnitine carrier (CAC) inhibitor and a peroxisomal beta oxidation inhibitor as active ingredients.

2. The pharmaceutical composition for the prevention or treatment of cancer of claim 1, wherein the carnitine acylcarnitine carrier inhibitor is at least one selected from the group consisting of omeprazole (KN510), lansoprazole (KN511), pantoprazole (KN512), and pharmaceutically acceptable salts thereof, and wherein the omeprazole is a compound represented by Formula 1 below:

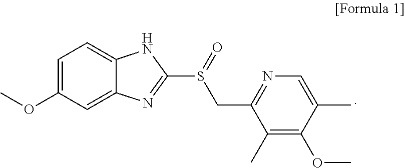

3. The pharmaceutical composition for the prevention or treatment of cancer of claim 1, wherein the peroxisome beta-oxidation inhibitor is thioridazine (KN714), represented by Formula 2 below, or a pharmaceutically acceptable salt thereof:

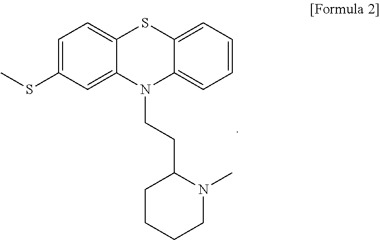

4. The pharmaceutical composition for the prevention or treatment of cancer of claim 1, wherein the composition comprises the carnitine acylcarnitine carrier inhibitor and peroxisome beta-oxidation inhibitor in a concentration ratio of 100:1 to 1:100.

5. The pharmaceutical composition for the prevention or treatment of cancer of claim 1, wherein the carnitine acylcarnitine carrier inhibitor and peroxisome beta-oxidation inhibitor are administered sequentially or simultaneously.

6. The pharmaceutical composition for the prevention or treatment of cancer of claim 1, wherein the cancer is one or more cancers selected from the group consisting of breast cancer, colon cancer, glioblastoma, liver cancer, leukemia, melanoma, lung cancer, ovarian cancer, prostate cancer, pancreatic cancer, renal cell carcinoma, and gastric cancer.

7. The pharmaceutical composition for the prevention or treatment of cancer of claim 1, wherein the composition further comprises an additional anticancer agent, and wherein the anticancer agent is one or more anti-cancer agents selected from one or more metabolism inhibitors selected from the group consisting of Irinotecan, Fluorouracil (5-FU), Paclitaxel, Gemcitabine, Cisplatin, Vermurafenib, and pharmaceutically acceptable salts thereof; and one or more tumor immunosuppressive agents selected from the group consisting of Pembrolizumab, Nivolumab, Atezolizumab, Ipilimumab, and Durvalumab.

8. An anti-cancer adjuvant comprising a carnitine acylcarnitine carrier (CAC) inhibitor and a peroxisomal beta oxidation inhibitor.

9. SThe anti-cancer adjuvant of claim 8, wherein the carnitine acylcarnitine carrier inhibitor is at least one selected from the group consisting of omeprazole (KN510), lansoprazole (KN511), pantoprazole (KN512), and pharmaceutically acceptable salts thereof, and wherein the omeprazole is a compound represented by Formula 1 below:

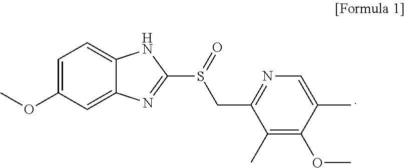

10. The anti-cancer adjuvant of claim 8, wherein the peroxisome beta-oxidation inhibitor is thioridazine (KN714), represented by Formula 2 below, or a pharmaceutically acceptable salt thereof:

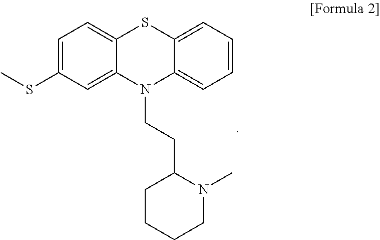

11. The anti-cancer adjuvant of claim 8, wherein the adjuvant comprises the carnitine acylcarnitine carrier inhibitor and the peroxisome beta-oxidation inhibitor in a concentration ratio of 100:1 to 1:100.

12. The anti-cancer adjuvant of claim 8, wherein the carnitine acylcarnitine carrier inhibitor and peroxisome beta-oxidation inhibitor are administered sequentially or simultaneously.

13. The anti-cancer adjuvant of claim 8, wherein the cancer is one or more cancers selected from the group consisting of breast cancer, colon cancer, glioblastoma, liver cancer, leukemia, melanoma, lung cancer, ovarian cancer, prostate cancer, pancreatic cancer, renal cell carcinoma, and gastric cancer.

14. The anti-cancer adjuvant of claim 8, wherein the adjuvant further comprises an additional anticancer agent, and wherein the anticancer agent is one or more anti-cancer agents selected from one or more metabolism inhibitors selected from the group consisting of Irinotecan, Fluorouracil (5-FU), Paclitaxel, Gemcitabine, Cisplatin, Vermurafenib, and pharmaceutically acceptable salts thereof; and one or more tumor immunosuppressive agents selected from the group consisting of Pembrolizumab, Nivolumab, Atezolizumab, Ipilimumab, and Durvalumab.

15. A method of preventing or treating cancer, comprising the step of administering or taking to a subject in need thereof a composition comprising a carnitine acylcarnitine carrier (CAC) inhibitor and a peroxisomal beta oxidation inhibitor.

16. (canceled).
